(19) United States
(12) Patent Application Publication
(10) Pub. No.: US 2024/0058618 A1
(43) Pub. Date: Feb. 22, 2024

(54) ULTRAVIOLET THERAPY APPARATUS AND METHOD FOR APPLYING ULTRAVIOLET LIGHT USING ULTRAVIOLET THERAPY APPARATUS

(71) Applicants: PUBLIC UNIVERSITY CORPORATION NAGOYA CITY UNIVERSITY, Nagoya-city, Aichi (JP); USHIO DENKI KABUSHIKI KAISHA, Tokyo (JP)

(72) Inventors: Akimichi MORITA, Aichi (JP); Hiroshi SHIBATA, Tokyo (JP); Tomohiko KIO, Tokyo (JP); Takashi HORIO, Tokyo (JP)

(73) Assignees: PUBLIC UNIVERSITY CORPORATION NAGOYA CITY UNIVERSITY, Nagoya-city, Aichi (JP); USHIO DENKI KABUSHIKI KAISHA, Tokyo (JP)

(21) Appl. No.: 18/259,473

(22) PCT Filed: Dec. 9, 2021
(86) PCT No.: PCT/JP2021/045411

§ 371 Date: Jun. 27, 2023

(30) Foreign Application Priority Data
Dec. 28, 2020 (JP) 2020-218331

(51) Int. Cl. A61N 5/06

(52) U.S. Cl. CPC A61N 5/06; A61N 2005/0627; A61N 2005/0651; A61N 2005/0661

(57) ABSTRACT

An ultraviolet therapy apparatus having an LED light source can achieve a stable therapeutic effect regardless of the temperature of the LED light source. The ultraviolet therapy apparatus includes an LED light source configured to emit light including ultraviolet light; a controller configured to control lighting of the LED light source; and a measurement unit configured to detect change in a temperature of the LED light source from a reference temperature. The controller includes a calculator configured to calculate a modifying value for modifying an irradiation dose of the ultraviolet light on the basis of change in a degree of effect on a human body caused by change in spectral spectrum of the light due to change in the temperature detected by the measurement unit, and a lighting controller configured to cause the LED light source to emit the light on the basis of the modifying value calculated by the calculator.

### TECHNICAL FIELD

The present invention relates to ultraviolet therapy apparatuses that use LEDs for light sources and methods for applying ultraviolet light using the ultraviolet therapy apparatuses.

### BACKGROUND ART

Conventional phototherapy includes ultraviolet therapy that uses ultraviolet light in the wavelength range of UVA (wavelength 320 nm to 400 nm) and UVB (wavelength 280 nm to 320 nm). Ultraviolet therapy is a treatment that uses ultraviolet light irradiation to achieve therapeutic effects for immunosuppression.

For example, Patent Document 1 (JP-A-2017-131522) discloses an ultraviolet therapy apparatus that treats skin diseases with ultraviolet light. This ultraviolet therapy apparatus includes a light source lamp or an LED as a source of ultraviolet light.

In a case in which LEDs are used for the light source, the circuit configuration therefor can be generally simpler than that for the power supply for the lamp, and the apparatus can be made smaller and lighter. For this reason, there have been proposed ultraviolet therapy apparatuses that use ultraviolet light emitting devices (UV LEDs) as the light sources for ultraviolet light.

In the following description, ultraviolet light and light containing ultraviolet light are sometimes simply referred to as “light.”

### BACKGROUND DOCUMENT

### Patent Document

Patent Document 1: JP-A-2017-131522

### SUMMARY OF THE INVENTION

### Problem to be Solved by the Invention

In contrast to light emitted from lamps, the wavelength and the irradiance of light emitted from LEDs vary depending on the temperature of the LEDs. In addition, when skin is irradiated with ultraviolet light, susceptibility to erythema, which is a side effect of ultraviolet light, differs depending on the wavelength. In other words, even if ultraviolet light is emitted for the same irradiation time from a single ultraviolet therapy apparatuses having an LED light source, susceptibility to the side effect may vary depending on the temperature of the LED light source.

Accordingly, it is an object of the present invention to provide an ultraviolet therapy apparatus having an LED light source that can achieve a stable therapeutic effect regardless of the temperature of the LED light source.

### Means for Solving the Problem

In accordance with an aspect of the present invention, there is provided an ultraviolet therapy apparatus, including an LED light source configured to emit light including ultraviolet light; a controller configured to control lighting of the LED light source; and a measurement unit configured to detect change in a temperature of the LED light source from a reference temperature. The controller includes a calculator configured to calculate a modifying value for modifying an irradiation dose of the ultraviolet light on the basis of change in a degree of effect on a human body caused by change in spectral spectrum of the light due to change in the temperature detected by the measurement unit, and a lighting controller configured to cause the LED light source to emit the light on the basis of the modifying value calculated by the calculator.

Thus, the change in temperature of the LED light source is monitored and the light irradiation dose is modified taking into account the change in the degree of effect on the human body (e.g., susceptibility to erythema) caused by the change in the spectral spectrum due to the change in temperature of the LED light source. Therefore, a stable therapeutic effect can be achieved in the same therapy apparatus, regardless of the temperature of the LED light source.

The ultraviolet therapy apparatus may include a storage unit for storing first information regarding an apparent irradiance that is obtained by modifying an irradiance on an irradiated surface of the ultraviolet light in view of an erythema action at each wavelength, and the calculator may calculate the modifying value using the first information stored in the storage unit.

In this case, modifying can be made appropriately using the apparent irradiance taking into account the erythema action at each wavelength.

In the above ultraviolet therapy apparatus, the first information may be information that indicates a relationship between a parameter correlated with the temperature of the LED light source and the apparent irradiance, and the calculator may be configured to derive a first modification factor that is a value obtained by dividing the apparent irradiance at the reference temperature by the apparent irradiance corresponding to the temperature of the LED light source on the basis of the change in temperature detected by the measurement unit with reference to the first information stored in the storage unit, and calculate the modifying value by multiplying a parameter that determines the irradiation dose of the ultraviolet light at the reference temperature by the first modification factor.

In this case, it is possible to estimate appropriately how much the apparent irradiance has changed from the apparent irradiance at the reference temperature because of the change in temperature of the LED light source from the reference temperature, and the reciprocal of the ratio of the apparent irradiance at the measured temperature to the apparent irradiance at the reference temperature can be derived as the first modification factor. By multiplying the first modification factor by the parameter that determines the irradiation dose of the ultraviolet light at the reference temperature, the above modifying value can be easily and appropriately calculated.

In the above ultraviolet therapy apparatus, the first information may be information that is useful for calculating the apparent irradiance and includes information on a spectral spectrum of the ultraviolet light and an erythema action spectrum, and the calculator may be configured to calculate the apparent irradiance at the temperature of the LED light source on the basis of the change in temperature detected by the measurement unit with reference to the first information stored in the storage unit, and calculate the modifying value on the basis of the calculated apparent irradiance.

In this case, the apparent irradiance at the measured temperature of the LED light source can be directly estimated and the above modifying value can be calculated appropriately.

The above ultraviolet therapy apparatus may include a storage unit for storing second information regarding an apparent irradiation dose derived from change in apparent irradiance during lighting of the LED light source, the apparent irradiance being obtained by modifying an irradiance on an irradiated surface of the ultraviolet light in view of an erythema action at each wavelength. The calculator may calculate the modifying value using the second information stored in the storage unit.

In this case, modifying can be made appropriately taking into account a fact that the apparent irradiance decreases as the temperature of the LED light source rises during lighting of the LED light source.

In the above ultraviolet therapy apparatus, the second information may be information that indicates a relationship between a determined irradiation dose to be applied to a patient and a ratio of the apparent irradiation dose to the determined irradiation dose. The ultraviolet therapy apparatus may further include an input unit configured to input the determined irradiation dose, and the calculator may be configured to derive a second modification factor that is a reciprocal of the ratio of the apparent irradiation dose to the determined irradiation dose on the basis of the determined irradiation dose inputted by the input unit with reference to the second information stored in the storage unit, and calculate the modifying value by multiplying a parameter that determines the irradiation dose of the ultraviolet light by the second modification factor.

In this case, it is possible to appropriately estimate how much the apparent irradiation dose will be relative to the determined irradiation dose, and to derive the reciprocal of the ratio of the apparent irradiation dose to the determined irradiation dose as the second modification factor. By multiplying the second modification factor by the parameter that determines the irradiation dose of the ultraviolet light, the above modifying value can be easily and appropriately calculated.

In the above ultraviolet therapy apparatus, the measurement unit may measure any one of a temperature of an LED substrate on which the LED light source is mounted, a forward voltage of the LED light source, and characteristics of the ultraviolet light from the LED light source. The spectral spectrum, irradiance, and radiant flux may be used as the characteristics of the light from the LED light source. In this case, change in the optical characteristics of the LED light source can be measured appropriately.

In the above ultraviolet therapy apparatus, the calculator may calculate, as the modifying value, any one of an irradiation time of the ultraviolet light, the inputted current for the LED light source, and a temperature adjustment amount for the LED light source. In this case, the parameter for modifying the light irradiation dose can be calculated appropriately.

In the above ultraviolet therapy apparatus, the measurement unit may detect the change in temperature of the LED light source before the LED light source is turned on by the lighting controller. The calculator may calculate an irradiation time of the ultraviolet light as the modifying value before the LED light source is turned on by the lighting controller, and the controller may have a display controller configured to display the light irradiation time calculated by the calculator on a display unit before the LED light source is turned on by the lighting controller.

In this case, the light irradiation time (treatment time) can be presented to users (doctor, patient, etc.) before the LED is turned on.

In accordance with an aspect of the present invention, there is provided a method for applying ultraviolet light using an ultraviolet therapy apparatus having an LED light source that emits light including ultraviolet light. The method includes a first step of detecting change in temperature of an LED light source from a reference temperature; a second step of calculating a modifying value for modifying an irradiation dose of the ultraviolet light on the basis of change in a degree of effect on a human body caused by change in spectral spectrum of the light due to the change in temperature; and a third step of causing the LED light source to emit on the basis of the modifying value.

Thus, the change in temperature of the LED light source is monitored and the light irradiation dose is modified taking into account the change in susceptibility to erythema caused by the change in the spectral spectrum due to the change in temperature of the LED light source. Therefore, a stable therapeutic effect can be achieved in the same therapy apparatus, regardless of the temperature of the LED light source.

### Effects of the Invention

According to the present invention, in an ultraviolet therapy apparatus using an LED (UV LED) light source that emits ultraviolet light, a stable therapeutic effect can be achieved, regardless of the temperature of the LED light source.

The objects, modes, and effects of the invention described above, as well as those not described above, will be understood by those skilled in the art from the following description of the mode of implementing the invention (detailed description of the invention) by referring to the accompanying drawings and the claims.

### BRIEF DESCRIPTION OF THE DRAWINGS

FIG. 1 is a graph showing the erythema action spectrum defined by the CIE;

FIG. 2 is a graph showing changes over time in a temperature of an LED substrate and the peak wavelength of the ultraviolet light;

FIG. 3 is a graph showing changes over time in a temperature of an LED substrate and relative irradiance;

FIG. 4 is a graph showing change in apparent irradiance over an irradiation time;

FIG. 5A is a graph for describing apparent irradiation doses:

FIG. 5B is a graph for describing a concept of modifying method 1;

FIG. 6A is a graph showing the relationship between the temperature of a substrate at the time of lighting and a ratio of change in apparent irradiance;

FIG. 6B is a graph showing a modification factor α;

FIG. 7 is a graph showing a concept of modifying method 2;

FIG. 8A is a graph showing the relationship between the determined irradiation dose and the ratio of the apparent irradiation dose to the determined irradiation dose;

FIG. 8B is a graph showing a modification factor β;

FIG. 9A is a graph showing effects modifying methods when the determined irradiation dose was 200 mJ/cm²;

FIG. 9B is a graph showing effect of modifying methods when the determined irradiation dose was 1500 mJ/cm²;

FIG. 10 is a flowchart showing a processing flow in an ultraviolet therapy apparatus according to an embodiment of the present invention; and

FIG. 11 is a block diagram of an example configuration of the ultraviolet therapy apparatus.

### DESCRIPTION OF EMBODIMENT

Hereinafter, with reference to the accompanying drawings, an embodiment of the present invention will be described.

As an embodiment, an ultraviolet therapy apparatus that includes a treatment tool that emits light containing ultraviolet light, for example, within the range of UVB (wavelength 280 nm to 320 nm) will be described. The ultraviolet therapy apparatus includes an LED light source manufactured so as to emit light having a peak at a wavelength of 308 nm as an example.

Exposure of human skin to ultraviolet light in the range of UVB causes erythema as a side effect. Erythema is redness of the skin surface caused by dilation of capillaries or other reasons. The dose of UVB irradiation after which a minimally perceptible skin erythema can be detected is referred to as the minimal erythema dose (MED). The unit of MED is mJ/cm². In precisely the same way as the susceptibility to sunburn varies among individuals, the susceptibility to erythema, or MED varies among individuals.

The susceptibility to ultraviolet erythema, i.e., the influence of ultraviolet on human bodies varies depending on the wavelength of the ultraviolet. The relative effectiveness on human bodies depending on the wavelength is defined by the International Commission on Illumination (CIE) as the erythema reference action spectrum.

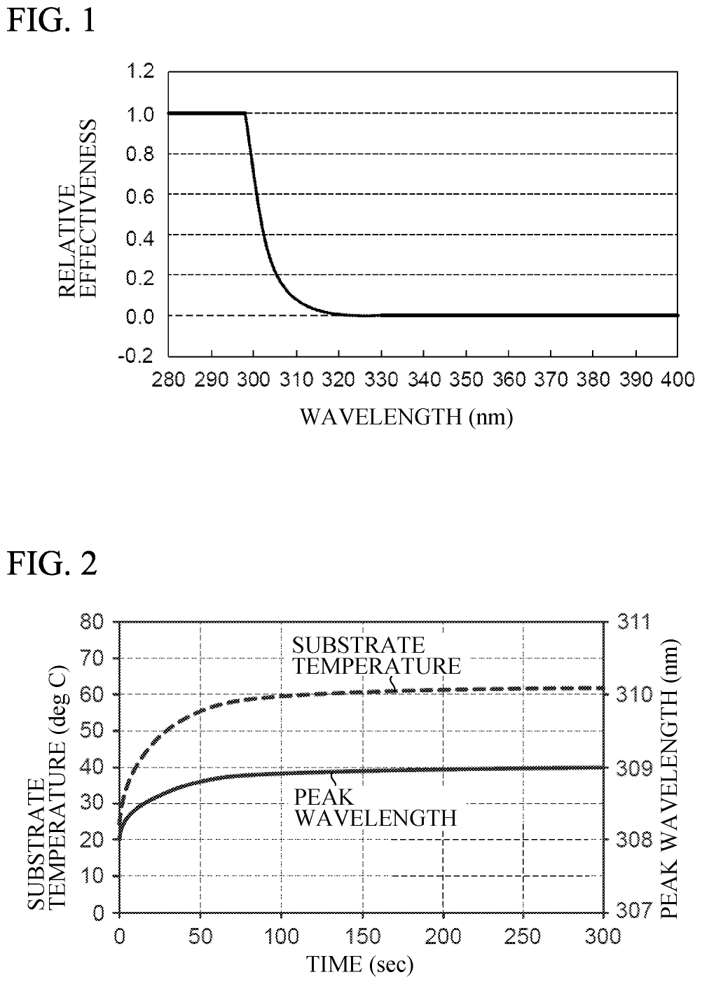

FIG. 1 is a graph showing the erythema action spectrum.

In FIG. 1, the horizontal axis indicates the wavelength (nm), and the vertical axis indicates the relative effectiveness. The erythema action spectrum Sₑᵣ is defined in the range of wavelength λ of 250 nm to 400 nm, and is defined as a relative effectiveness depending on wavelengths as in Formula (1), in which the relative effectiveness is a relative value on the assumption that the influence of light with a wavelength of 250 nm to 298 nm on the human skin is one.

$${S}_{er} ( λ ) = \{ \begin{matrix}1 & ( when 250 nm < λ < 298 nm ) \\ {10}^{0.094 ( 298 - λ )} & ( when 298 nm \leq λ \leq 328 nm ) \\ {10}^{0.015 ( 139 - λ )} & ( when 328 nm \leq λ < 400 nm )\end{matrix} \qquad ( 1 )$$

From the outline of the graph shown in FIG. 1, it can be understood that shorter wavelengths have a greater impact on human bodies and are more likely to cause erythema. Specifically, light with wavelengths longer than the UVB region, or longer than the wavelength of 328 nm (in a case in which Formula (1) is strictly applied), has little impact on the skin. On the other hand, light with wavelengths below 328 nm affects the skin, and this effect increases as the wavelength is shorter.

The overall effect of ultraviolet radiation on the human body is obtained by integrating the product of the spectral irradiance E_{λ} of the ultraviolet and the erythema action spectrum Sₑᵣ in the wavelength range of 250 nm to 400 nm as in Formula (2). The effect thus obtained is called the erythemal ultraviolet irradiance I_{CIE}. The higher the value of the erythemal ultraviolet irradiance I_{CIE} is, the more likely ultraviolet is to cause erythema.

I_{CIE}=∫₂₅₀⁴⁰⁰E_{λ}×Sₑᵣdλ (2)

As can be seen from FIG. 1, especially in the wavelength range of 298 nm to 310 nm, even only a one nanometer change in the wavelength causes a significant change in the degree of effect on a human body.

It is generally known that optical characteristics of LED light sources vary depending on the temperature thereof.

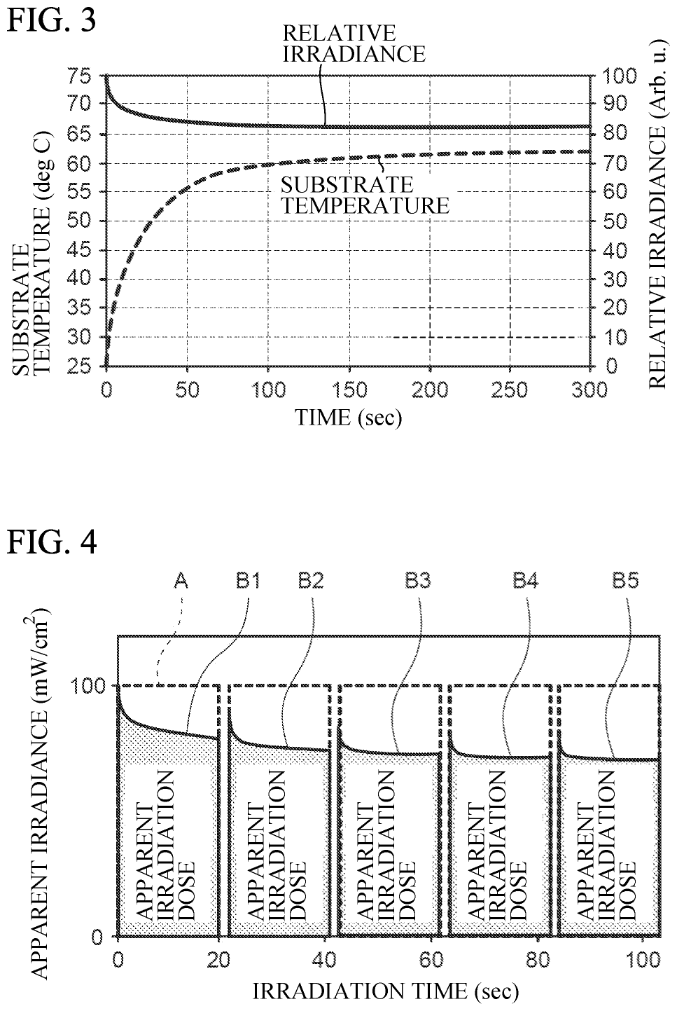

FIG. 2 is a graph showing changes over time in a temperature of a substrate for an LED light source and the peak wavelength of the ultraviolet light on an irradiated surface after lighting the LED light source. FIG. 3 is a graph showing changes over time in a temperature of another substrate for an LED light source and relative irradiance on an irradiated surface after lighting the LED light source. Since it is not possible to measure the temperature of the LED light source itself, the temperature of the LED substrate, which correlates with the temperature of the LED light source was measured.

As shown in FIGS. 2 and 3, as the temperature of the LED light source (and thus, the temperature of the substrate) increases due to energization after turning on the LED light source, the wavelength of the emitted ultraviolet light becomes longer and the irradiance on the irradiated surface decreases. This result means that the ultraviolet light emitted from the single LED light source becomes less likely to cause erythema as time advances.

In other words, even when the same ultraviolet therapy apparatus is used for a therapeutic treatment for the same irradiation time, the appearance of the side effect and/or the degree of the therapeutic effect may vary depending on the temperature of the LED light source.

In the embodiment, such changes in the optical characteristics of an LED light source are monitored and the ultraviolet irradiation dose is modified so that the therapeutic effect achieved by the ultraviolet therapy apparatus does not change (so that a desired ultraviolet irradiation dose is provided). Here, it will be described that the irradiation time, which is a parameter that determines the ultraviolet irradiation dose, is modified on the basis of the temperature of the LED substrate, which reflects the LED optical characteristics, so that the ultraviolet irradiation dose is modified.

The following is a specific description of how the spectral spectrum variation of the LED light source influences the therapeutic treatment.

As described above, the wavelength of ultraviolet light emitted from an LED light source becomes longer as the temperature of the LED light source rises. Therefore, in an LED-type ultraviolet therapy apparatus, the emitted light becomes less likely to result in erythema over time as if the irradiance on the irradiated surface decreases in appearance. Accordingly, an “apparent irradiance” is defined as an index that is obtained by modifying the irradiance on the irradiated surface in view of the erythema susceptibility at each wavelength as in Formula (3).

$$\begin{matrix}{E}^{′} ( T ) = \frac{{I}_{CIE} ( T )}{{I}_{CIE} ( {T}_{0} )} E ( {T}_{0} ) \\ = \frac{\int {E}_{λ} ( T , λ ) \cdot {S}_{er} ( λ ) d λ}{\int {E}_{λ} ( {T}_{0} , λ ) \cdot {S}_{er} ( λ ) d λ} \cdot E ( {T}_{0} ) \\ = \frac{\int P ( T , λ ) \cdot E ( T ) \cdot {S}_{er} ( λ ) d λ}{\int P ( {T}_{0} , λ ) \cdot E ( {T}_{0} ) \cdot {S}_{er} ( λ ) d λ} \cdot E ( {T}_{0} ) \\ = \frac{\int P ( T , λ ) \cdot {S}_{er} ( λ ) d λ}{\int P ( {T}_{0} , λ ) \cdot {S}_{er} ( λ ) d λ} \cdot \frac{E ( T )}{E ( {T}_{0} )} \cdot E ( {T}_{0} )\end{matrix} \qquad ( 3 )$$

In Formula (3), F(T) is the apparent irradiance at a temperature T of the LED substrate. E(T) is the actual irradiance on the irradiated surface at a temperature T of the LED substrate. T is a temperature of the LED substrate. T₀ is a reference temperature of the LED substrate (e.g., 25 degrees Celsius). Furthermore, I_{CIE}(T) is the erythemal ultraviolet irradiance for the emitted light at the LED substrate temperature T, and I_{CIE}(T₀) is the erythemal ultraviolet irradiance for the emitted light at the reference LED substrate temperature T₀. E_{λ}(T,λ) is the spectral irradiance at the LED substrate temperature T. Sₑᵣ(λ) is the erythema action spectrum. P(T,λ) is an area-normalized spectral spectrum.

Thus, the apparent irradiance E′(T) at a temperature T is calculated by modifying the irradiance E(T₀), which is an initial irradiance at the reference temperature T₀, taking into account change in erythema action due to change in spectral spectrum caused by change in temperature of the LED substrate, and change in actual irradiance caused by change in temperature of the LED substrate. The change in actual irradiance caused by change in temperature of the LED substrate (corresponding to E(T)/E(T₀)) can also be obtained from a parameter correlated with the irradiance (e.g., an integral value of the spectral spectrum within a wavelength range).

In a case in which the light emitted from the LED light source contains light with a wavelength range of 298 nm to 400 nm, the apparent irradiance decreases monotonically as the LED substrate temperature increases. Accordingly, if the irradiation time is determined using only the initial irradiance in an ultraviolet therapy apparatus without considering the relationship between the LED substrate temperature and the apparent irradiance, an appropriate irradiation dose cannot be obtained.

Let us consider a phase in which an actual therapeutic treatment is conducted. In an ultraviolet therapy apparatus, the irradiation time was automatically calculated on the basis of the determined irradiation dose input into the apparatus by the user. In a conventional ultraviolet therapy apparatus, the irradiation time was calculated by dividing the determined irradiation dose input by the user by an irradiance value (initial irradiance) that is preset in the therapy apparatus.

For example, let us consider a case of light irradiation at 2000 mJ/cm². If the initial irradiance is 100 mW/cm² and an ideal light source of which the irradiance does not change over time, the irradiation time is calculated as 20 seconds. However, as mentioned above, the apparent irradiance decreases after the LED light source is turned on. Accordingly, even though the apparent irradiance when the LED light source is turned on is the initial irradiance (100 mW/cm²), if light irradiation is conducted for the irradiation time estimated from the initial irradiance, the irradiation dose will be insufficient.

Next, let us consider a case in which the determined irradiation dose is 2000 mJ/cm² and the irradiation is conducted in five cycles in a row. The irradiation cycle time is assumed to be 20 seconds according to the above calculation, and the interval between the cycles is assumed to be 1 second. Measurement results of the apparent irradiance under these conditions are shown in FIG. 4.

In FIG. 4, the dotted-line frames A depict the irradiance when an ideal light source is used (determined irradiance), whereas the solid-line frames B1 to B5 depict the apparent irradiances at the first to the fifth cycles. FIG. 4 shows that the apparent irradiance from the LED light source decreased over time during each cycle, and that the apparent irradiance at the start of LED lighting decreased as the number of irradiation cycles increased. As a result, it will be understood that the difference between the determined irradiation dose and the apparent irradiation dose (irradiation dose error) increased as the number of irradiation cycles increased, and that proper irradiation could not be achieved, especially at the end of the multiple irradiation cycles. This means that the LED-type ultraviolet therapy apparatus cannot provide a constant therapeutic effect since the apparent irradiation dose varies from irradiation cycle to irradiation cycle even if the irradiation cycle time is uniform.

Modifying Method 1

Since the apparent irradiance varies depending on the temperature of the LED substrate, the inventors conceived of a modifying method in which the temperature of the LED substrate is obtained at the start of each irradiation cycle (when an irradiation switch is pressed), and the apparent irradiance is estimated from the obtained temperature of the LED substrate to modify the irradiation time. This modifying method will be called “modifying method 1.

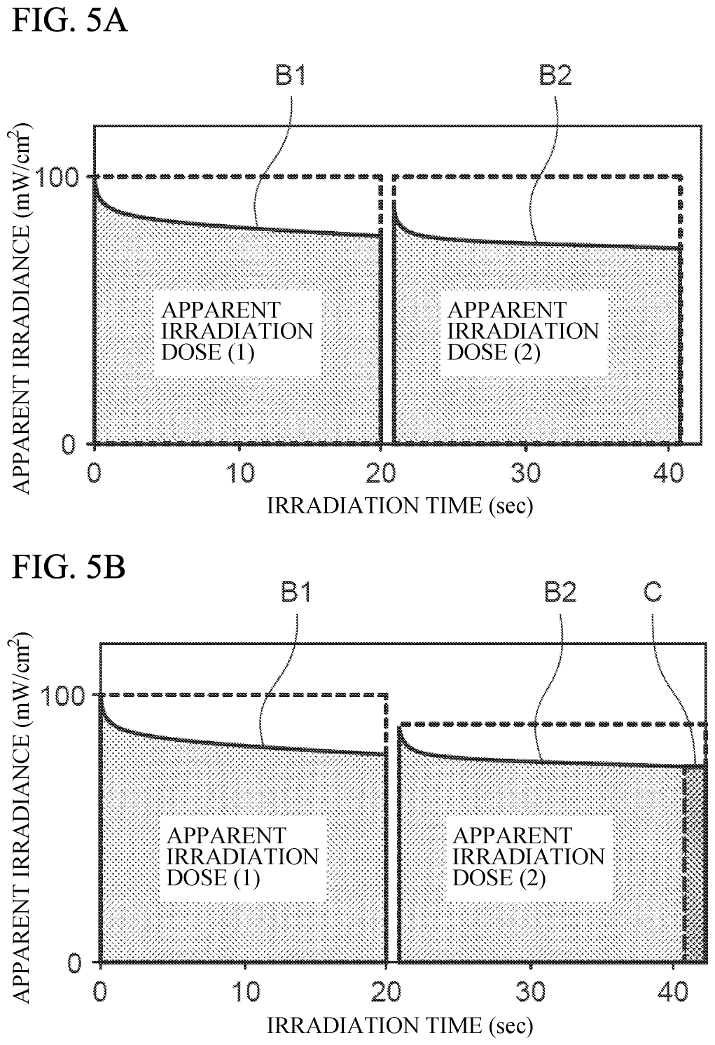

As shown in FIG. 5A, in a case in which the irradiation is conducted in multiple cycles (two cycles in this case) in a row with the same irradiation cycle time, the apparent irradiance at the start of irradiation in the second cycle is lower since the temperature of the LED substrate in the second cycle is higher than that in the first cycle. Therefore, the apparent irradiation dose B2 in the second irradiation cycle is less than the apparent irradiation dose B1 in the first irradiation cycle.

Accordingly, the irradiation time is modified in view of the decrease in the apparent irradiance. Specifically, the temperature of the LED substrate is obtained at the start of each irradiation cycle, and the apparent irradiance at the start of each irradiation cycle is estimated, and an additional irradiation time is added in view of the value of the apparent irradiance, as shown in FIG. 5B. As a result, the apparent irradiation dose in the second irradiation cycle is increased by the additional amount C caused by the modification of the irradiation time, and therefore, the difference between the apparent irradiation dose in the first irradiation cycle and that in the second irradiation cycle becomes less, that is, the variation of the apparent irradiation dose in each irradiation cycle is expected to be less.

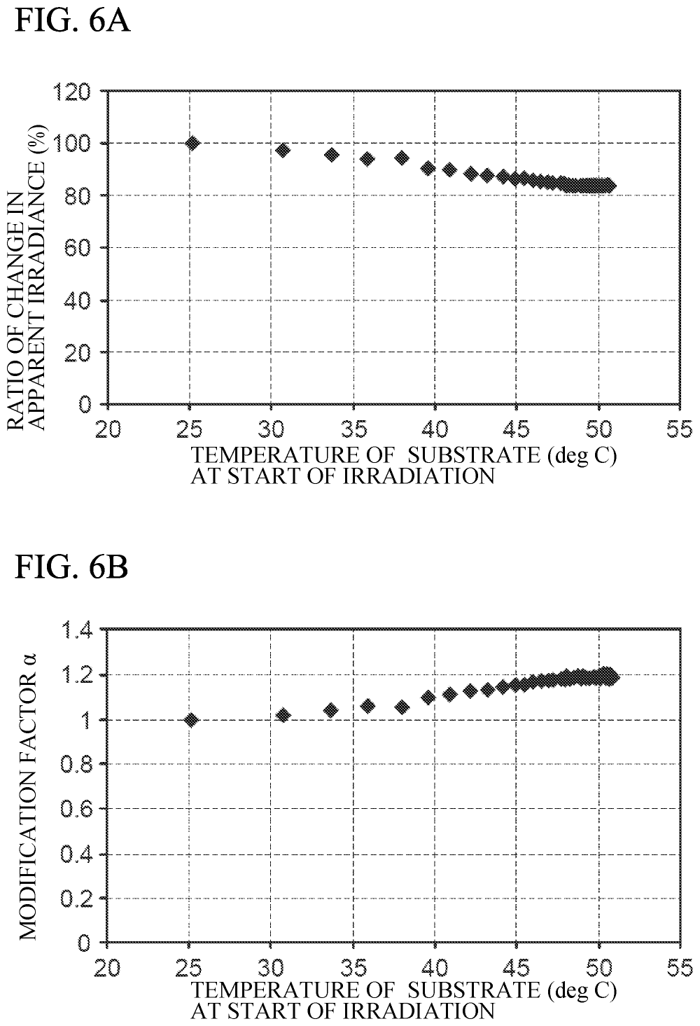

FIG. 6A shows the relationship between the temperature of an LED substrate and the apparent irradiance. In FIG. 6A, the vertical axis indicates the ratio of change in apparent irradiance, normalized by the substrate temperature of 25 degrees Celsius. That is to say, the ratio of change in apparent irradiance is the ratio of the apparent irradiance at a temperature T of the LED substrate to the apparent irradiance at the temperature of the LED substrate is the reference temperature (25 degrees Celsius).

In the embodiment, the reciprocal of the ratio of change in apparent irradiance shown in FIG. 6A (the value obtained by dividing the apparent irradiance when the temperature of the LED substrate is the reference temperature, 25 degrees Celsius, by the apparent irradiance at a temperature T of the LED substrate) is used for a modification factor α, and the irradiation time is modified by multiplying the irradiation time by the modification factor α. FIG. 6B shows the relationship between the temperature of the LED substrate and the modification factor α. The modification factor α shown in FIG. 6B is stored in the ultraviolet therapy apparatus as a function or a matrix (table).

For example, the modification factor α is 1.00 when the temperature of the LED substrate is 25 degrees Celsius, and the modification factor α is 1.20 when the temperature of the LED substrate is 50 degrees Celsius.

Accordingly, for example, in a case in which the determined irradiation dose is 200 mJ/cm² for an ultraviolet therapy apparatus from which the irradiance is 100 mW/cm² at the reference temperature (25 degrees Celsius) of the LED substrate, when the temperature of the LED substrate is 25 degrees Celsius, the irradiation time is 2.00 sec, which is equal to 200/100×1.00. In addition, when the temperature of the LED substrate is 50 degrees Celsius, the irradiation time is 2.40 sec, which is equal to 200/100×1.20.

In this way, the change in optical characteristics (change in apparent irradiance) is estimated on the basis of the temperature of the LED substrate at the start of each irradiation cycle, and the irradiation time is modified for each irradiation cycle.

Modifying Method 2

With LED light sources, the apparent irradiance decreases monotonically as the temperature of the LED substrate rises after lighting. Accordingly, it is considered that as the determined irradiation dose increases (as the irradiation time increases), the irradiation dose error also increases. The inventors conceived of a modifying method in which the irradiation time is modified depending on the determined irradiation dose. This modifying method will be called “modifying method 2.

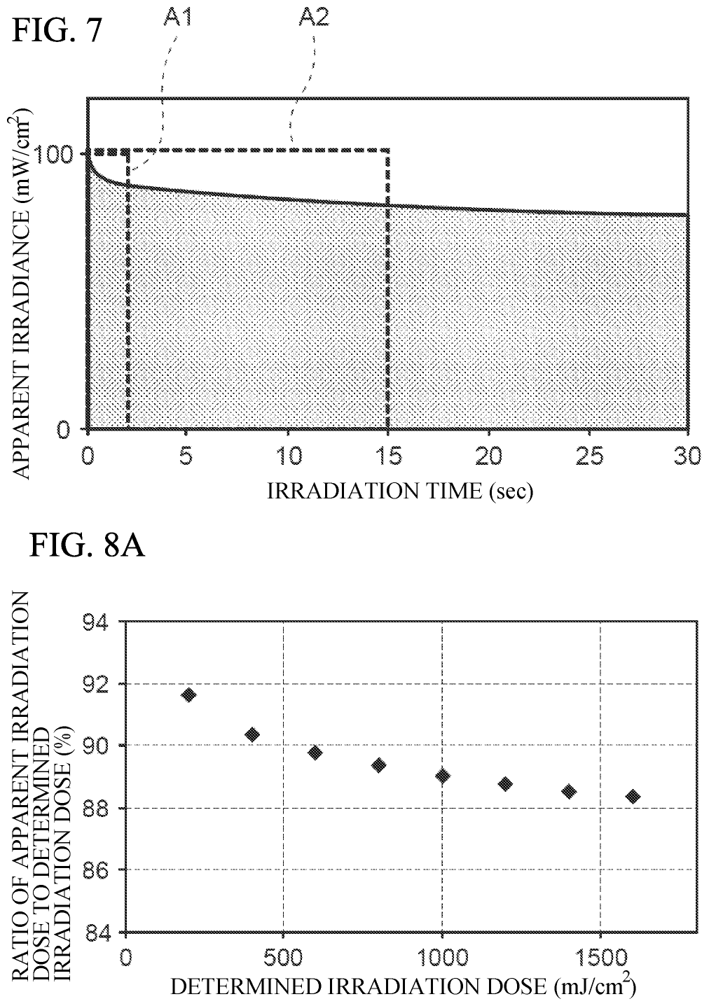

For example, let us consider a case in which the determined irradiation dose is 200 mJ/cm² and another case in which the determined irradiation dose is 1500 mJ/cm² in a therapy apparatus with an initial irradiance of 100 mW/cm². A conceptual diagram is shown in FIG. 7.

In FIG. 7, the dotted-line frame A1 depicts the determined irradiation dose of 200 mJ/cm² and the dotted-line frame A2 depicts the determined irradiation dose of 1500 mJ/cm². As shown in FIG. 7, the apparent irradiance decreases as time advances. In addition, the irradiation time becomes longer as the determined irradiation dose becomes larger. Therefore, the greater the determined irradiation dose is, the greater the irradiation dose error is.

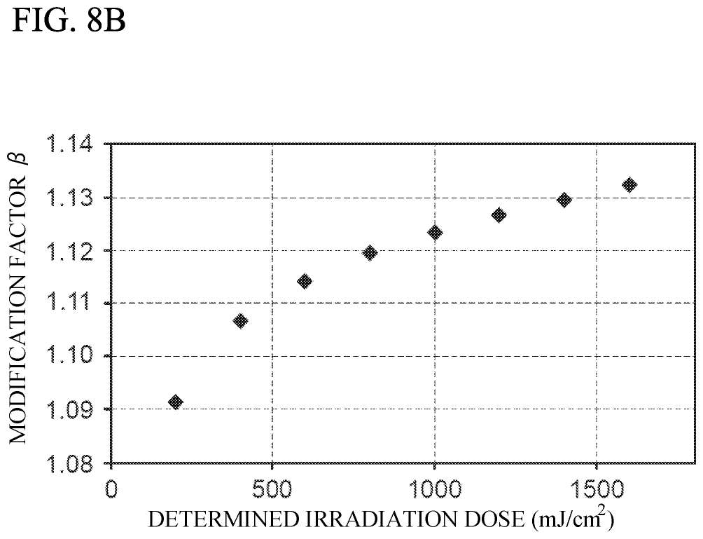

FIG. 8A shows the relationship between the determined irradiation dose and the ratio of the apparent irradiation dose to the determined irradiation dose. Here, the apparent irradiation dose is calculated as the integral value of the apparent irradiance over the irradiation time as will be understood by the curve shown in FIG. 7. As shown in FIG. 8A, the greater the determined irradiation dose is, the less the ratio of the apparent irradiation dose to the determined irradiation dose is.

Accordingly, in the embodiment, the reciprocal of the ratio of the apparent irradiation dose to the determined irradiation dose shown in FIG. 8A is used for a modification factor β, and the irradiation time is modified by multiplying the irradiation time by the modification factor β. FIG. 8B shows the relationship between the determined irradiation dose and the modification factor β. The modification factor β shown in FIG. 8B is stored in the ultraviolet therapy apparatus as a function or a matrix (table).

For example, the modification factor β is 1.09 when the determined irradiation dose is 200 mJ/cm², and the modification factor β is 1.13 when the determined irradiation dose is 1500 mJ/cm².

Accordingly, for example, for an ultraviolet therapy apparatus from which the irradiance is 100 mW/cm² at the reference temperature (25 degrees Celsius) of the LED substrate, when the determined irradiation dose is 200 mJ/cm², the irradiation time is 2.18 sec, which is equal to 200/100×1.09. In addition, when the determined irradiation dose is 1500 mJ/cm², the irradiation time is 17.0 sec, which is equal to 1500/100×1.13.

In this way, the irradiation time is modified depending on the determined irradiation dose.

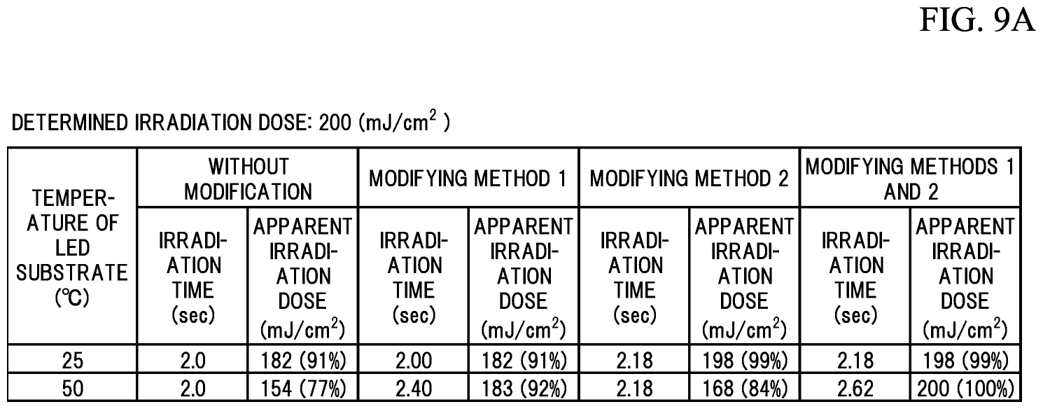

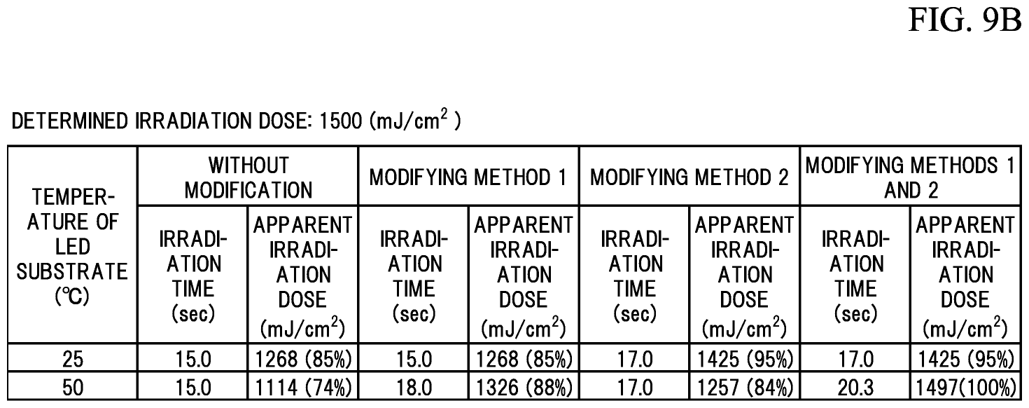

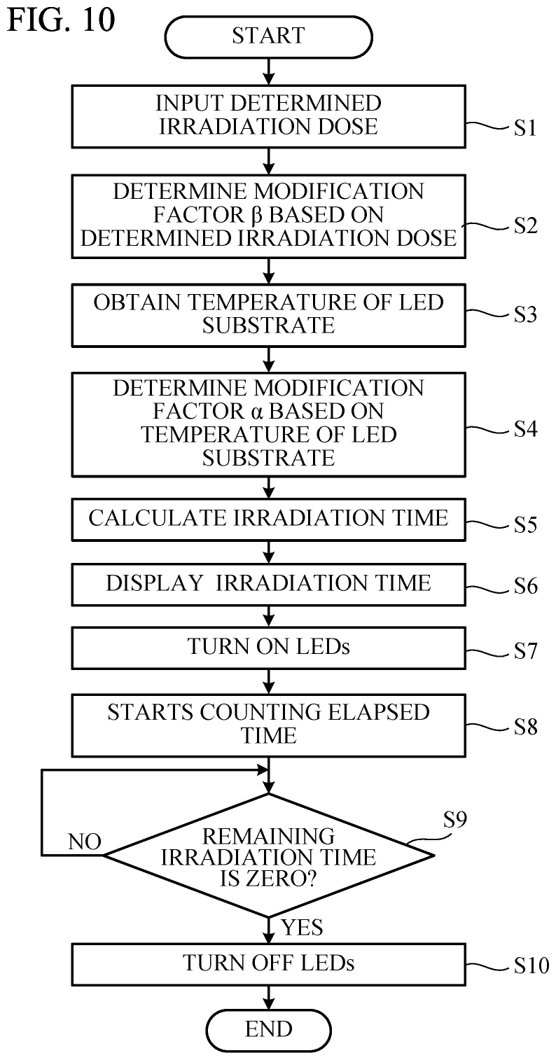

The inventors confirmed effects of modifications for cases in which only modifying method 1 was executed, only modifying method 2 was executed, and both modifying methods 1 and 2 were executed. The results are shown in FIGS. 9A and 9B.

FIG. 9A shows the effects in which the determined irradiation dose was 200 mJ/cm², and FIG. 9B shows the effects in which the determined irradiation dose was 1500 mJ/cm². At each determined irradiation dose, the irradiation time was modified in cases in which the temperatures of the LED substrate were 25 degrees Celsius and 50 degrees Celsius, and the results were compared. The values in parentheses in FIGS. 9A and 9B are the ratio of the apparent irradiation dose to the determined irradiation dose.

In the case in which only modifying method 1 was implemented, the irradiation time was not modified when the temperature of the LED substrate was 25 degrees Celsius, which is the reference temperature. On the other hand, when the temperature of the LED substrate was 50 degrees Celsius, the irradiation time was modified (added) to compensate for the decrease in apparent irradiance. As a result, the variation of apparent irradiance caused by the temperature difference was reduced at the same determined irradiation dose. In other words, when the determined irradiation dose was the same, almost the same therapeutic effect was obtained regardless of the temperature of the LED substrate.

However, the irradiation dose errors differed depending on the determined irradiation dose. Specifically, when the determined irradiation dose was 1500 mJ/cm², the irradiation dose error was greater than that when the determined irradiation dose was 200 mJ/cm². Thus, the problem of the irradiation dose errors being different depending on the determined irradiation dose could not be solved by implementing only modifying method 1.

On the other hand, in the case in which only modifying method 2 was implemented, the irradiation time was modified depending on the determined irradiation dose. As a result, under the same temperature conditions, the irradiation dose errors were almost the same regardless of the determined irradiation dose. However, in the case in which only modifying method 2 was implemented, the difference in irradiation dose error caused by the difference in the LED substrate temperature at the start of irradiation could not be modified.

In the case in which modifying method 1 was combined with the modifying method 2, the apparent irradiation dose could be closer to the determined irradiation dose in both cases shown in FIGS. 9A and 9B, regardless of the temperature of the LED substrate at the time of irradiation start and the determined irradiation dose. Thus, by implementing modifying method 1 in combination with modifying method 2, an irradiation dose that should be applied to a patient could be appropriately provided.

A processing flow when using an LED-type ultraviolet therapy apparatus according to an embodiment of the present invention will be described with reference to FIG. 10.

In the ultraviolet therapy apparatus, the irradiance E [mW/cm²] on an irradiated surface at the reference temperature (25 degrees Celsius) measured in advance, for example, prior to shipment and information for deriving the modification factors α and β (FIGS. 6B and 8B) are stored. An ultraviolet therapy apparatus using an LED light source generally includes multiple LEDs (e.g., 5×5 LED array). The above-mentioned irradiance is the irradiance of the composite light emitted from the multiple LEDs.

At step S1, the ultraviolet therapy apparatus obtains the determined irradiation dose H [mJ/cm²] determined and input by a physician according to the patient's disease, and the process proceeds to step S2.

At step S2, the ultraviolet therapy apparatus refers to a table corresponding to FIG. 8B using the determined irradiation dose H obtained at step S1 as a key, and derives the modification factor β.

At step S3, the ultraviolet therapy apparatus obtains the temperature T of the LED substrate.

At step S4, the ultraviolet therapy apparatus refers to a table corresponding to FIG. 6B using the temperature T of the LED substrate obtained at step S3 as a key, and derives the modification factor α.

At step S5, the ultraviolet therapy apparatus calculates the irradiation time t as a modifying value for modifying the determined irradiation dose of light. Specifically, the ultraviolet therapy apparatus calculates the irradiation time t (sec) by dividing the determined irradiation dose H by the irradiance E stored in the ultraviolet therapy apparatus to produce a quotient and multiplying the quotient by the modification factors α and β.

t=H/E×α×β (4)

At step S6, the ultraviolet therapy apparatus controls a display unit provided therein to display the irradiation time t calculated at step S5.

At step S7, the LEDs start irradiation when a switch provided in the ultraviolet therapy apparatus is pressed by the physician or, in some cases, a nurse or other medical service worker.

At step S8, the ultraviolet therapy apparatus starts counting the elapsed time.

At step S9, the ultraviolet therapy apparatus determines whether or not the remaining irradiation time has reached 0. If the ultraviolet therapy apparatus determines that the remaining irradiation time is not 0, it continues to cause the LEDs to be energized. If it determines that the remaining irradiation time is 0, the process proceeds to step S10 in which the LEDs are turned off. In this way, the therapeutic treatment is conducted for the irradiation time of t seconds.

Thus, based on the determined irradiation dose H [mJ/cm²] input by the physician and the temperature T of the LED substrate [degrees Celsius] at the start of irradiation, an appropriate irradiation time t in view of the change in the optical characteristics of the LED light source can be calculated.

By conducting the therapeutic treatment for the irradiation time t calculated by the therapy apparatus, it is possible to reduce variation of the degree of the therapeutic effect and/or the appearance of the side effect from irradiation to irradiation, and to reduce a situation that the determined irradiation dose is not provided so that a sufficient therapeutic effect is not achieved.

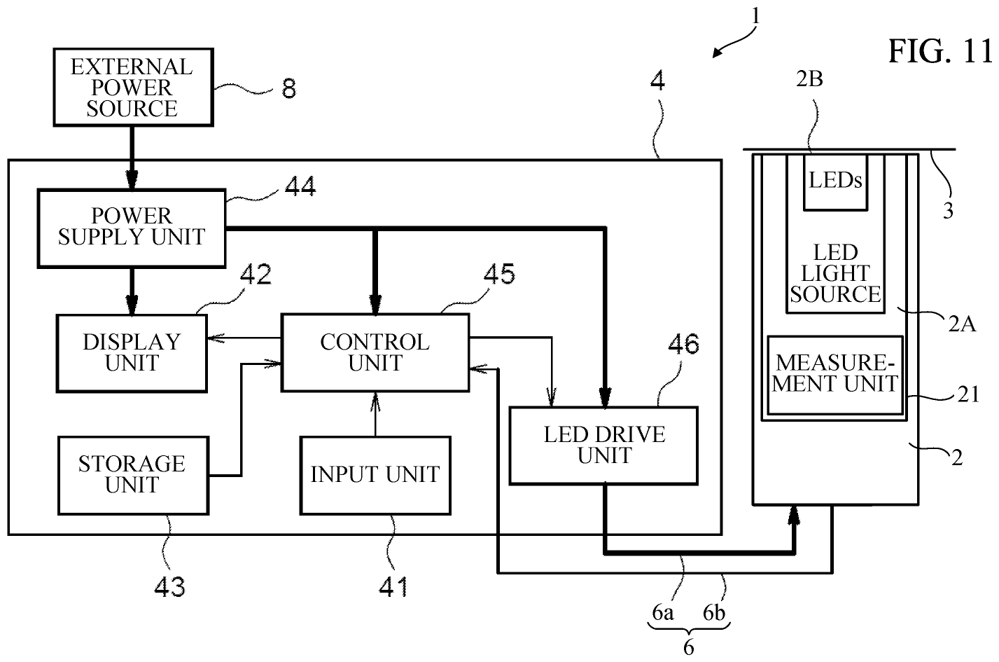

FIG. 11 is a block diagram showing an example configuration of the ultraviolet therapy apparatus 1 according to an embodiment of the present invention.

The ultraviolet therapy apparatus 1 includes a treatment tool (light source part) 2 that has an LED light source that emits light including ultraviolet light, and a main unit 4 that controls the LED light source of the treatment tool 2.

The treatment tool 2 includes a measurement unit 21 that measures the temperature of the LED substrate. The measurement unit 21 has a structure in which a temperature measuring probe, such as a thermistor or thermocouple, is mounted on the LED substrate.

The main unit 4 includes an input unit 41, a display unit 42, a storage unit 43, a power supply unit 44, a control unit (controller) 45, and an LED drive unit 46. The treatment tool 2 and the main unit 4 are connected by a connection cable 6, which has a power line 6a shown by a thick line and a signal line 6b shown by a thin line.

The input unit 41 obtains the determined irradiation dose H input by an operator (e.g., the physician) and outputs the information on the determined irradiation dose H to the control unit 45.

The display unit 42 can display the UV irradiation intensity, the irradiation time, the elapsed time during ultraviolet irradiation, and other information. The display unit 42 can also display information (such as an error message) indicating that an abnormality has occurred if some abnormality occurs in the ultraviolet therapy apparatus 1.

The storage unit 43 stores the irradiance E on the irradiated surface of the ultraviolet therapy apparatus 1 and information for deriving the modification factors α and β.

The power supply unit 44 adjusts the voltage of the electric power supplied from an external power source 8 to an appropriate level and supplies the power to each of the units in the subsequent stages.

The control unit 45 acquires the temperature T of the LED substrate measured by the measurement unit 21 and the determined irradiation dose H input into the input unit 41, and derives the modification factors α and β on the basis of them. The control unit 45 modifies the irradiation time that is obtained by dividing the determined irradiation dose H by the irradiance E stored in the storage unit 43 and calculates a modified irradiation time t using the modification factors α and R. The control unit 45 then controls the LED drive unit 46 to control the irradiation amount (irradiation time t) of the LED light source in the treatment tool 2. In other words, the control unit 45 has a calculator that calculates a modifying value (in this case, the irradiation time) for modifying the irradiation dose and a lighting controller that causes the LED light source to emit the light on the basis of the modifying value.

The LED drive unit 46 supplies electric power to the LED light source in accordance with the control signal from the control unit 45.

Hereinafter, a procedure in which an operator applies ultraviolet to an affected area using the ultraviolet therapy apparatus 1 of the embodiment will be described.

First, the operator manipulates the input unit 41 to input the ultraviolet irradiation dose (determined irradiation dose H) for irradiating the affected area.

Next, the operator holds the treatment tool 2 and brings a light-outputting surface thereof, through which the light from the LED light source is outputted, in contact with or close to the affected area. The operator then presses a switch (not shown) provided in the treatment tool 2. Then, the temperature T of the LED substrate is measured in the ultraviolet therapy apparatus 1, and the ultraviolet light irradiation time t is calculated depending on the temperature T of the LED substrate and the determined irradiation dose H. The calculated irradiation time t is displayed on the display unit 42. Subsequently, the LED light source is turned on to start the ultraviolet irradiation to the affected area.

Thereafter, when the irradiation time reaches the calculated irradiation time t, the LED light source is automatically turned off.

As described above, the ultraviolet therapy apparatus 1 in the embodiment includes the treatment tool 2 having the LED light source that emits light including ultraviolet light, the control unit (controller) 44 that controls lighting of the LED light source, and the measurement unit 21 that measures the temperature of the LED substrate, thereby detecting change in temperature of the LED light source from the reference temperature. The control unit 45 calculates a modifying value for modifying a light irradiation dose on the basis of change in a degree of effect on a human body caused by change in spectral spectrum of the light due to the change in temperature of the LED light source (change in temperature of the LED substrate), and causes the LED light source to emit the light on the basis of the calculated modifying value. Here, the above-mentioned modifying value can be the light irradiation time, which is a parameter that determines the light irradiation dose.

Specifically, the ultraviolet therapy apparatus 1 includes the storage unit 43 that stores first information regarding the apparent irradiance, which is obtained by modifying the irradiance on the irradiated surface of the light in view of the erythema action at each wavelength. The first information is information that indicates the relationship between a parameter correlated with the temperature of the LED light source and the apparent irradiance. For example, the first information may be information that associates the temperature of the LED substrate with the modification factor α (first modification factor) shown in FIG. 6B. The modification factor α is the reciprocal of the ratio of change in apparent irradiance shown in FIG. 6A, and is a value obtained by dividing the apparent irradiance at the reference temperature (25 degrees Celsius) by the apparent irradiance at the measured temperature.

The control unit 45 derives the modification factor α on the basis of the temperature of the LED substrate with reference to the first information and modifies the irradiation time by multiplying a parameter that determines the light irradiation dose at the reference temperature (irradiation time H/E at the reference temperature) by the modification factor α (modifying method 1).

The storage unit 43 may also store second information regarding the apparent irradiance derived from the change in apparent irradiance during LED light source lighting. The second information is information that indicates the relationship between the determined irradiation dose to be applied to the patient and the ratio of the apparent irradiation dose to the determined irradiation dose. For example, the second information may be information that associates the determined irradiation dose with the modification factor β (second modification factor) shown in FIG. 8B. The modification factor β is the reciprocal of the ratio of the apparent irradiation dose to the determined irradiation dose shown in FIG. 8A.

The control unit 45 derives the modification factor β on the basis of the inputted determined irradiation dose with reference to the second information and modifies the irradiation time by multiplying the parameter that determines the light irradiation dose (in the embodiment, the irradiation time H/E× α after modifying with use of the temperature of the LED substrate) by the modification factor β (modifying method 2).

The control unit 45 then causes the LED light source to emit the ultraviolet for the modified irradiation time t.

In summary, a method for applying ultraviolet light using the ultraviolet therapy apparatus 1 in the embodiment includes a first step of detecting change in temperature of an LED light source from a reference temperature; a second step of calculating a modifying value for modifying a light irradiation dose on the basis of change in a degree of effect on a human body caused by change in spectral spectrum of the light due to the change in temperature of the LED light source; and a third step of causing the LED light source to emit on the basis of the calculated modifying value.

In this way, the change in temperature of the LED light source is monitored and the light irradiation dose is modified taking into account the change in the susceptibility to erythema caused by the change in the spectral spectrum due to the change in temperature of the LED light source. In other words, the modifying method takes into account not only change in actual irradiance caused by the change in temperature of the LED light source, but also the effect of change in wavelength. As described above, in ultraviolet therapy apparatuses, even only a one nanometer change in the wavelength causes a significant change in the degree of therapeutic effect on a human body. According to the ultraviolet therapy apparatus 1 in the embodiment, a stable therapeutic effect can be achieved in the same therapy apparatus, regardless of the temperature of the LED light source.

The ultraviolet therapy apparatus 1 in the embodiment is a compact therapy apparatus using LEDs for the light source. Therefore, when the therapeutic treatment is desired for a wide range of affected areas, the therapeutic light cannot be applied to the affected areas in a single light irradiation, and therefore, multiple light irradiation cycles may be necessary with changing the irradiation position. In this case, if the therapeutic effect is not the same in each irradiation cycle, uneven treatment results will occur.

As described above, by applying modifying method 1, in which the irradiation time is modified in view of the temperature of the LED light source, the same irradiation dose can be applied as long as the determined irradiation dose is the same, even if the temperature of the LED light source changes due to multiple irradiation cycles in a row. Accordingly, the same therapeutic effect can be achieved in each irradiation cycle. In addition, when light irradiation is conducted in multiple cycles, there is no need to wait for dropping the temperature of the LED light source to the reference temperature before restarting light irradiation in order to obtain the same therapeutic effect in each cycle. This is time-efficient.

On the other hand, the degree of skin diseases differs among individuals, and the desirable irradiation dose (determined irradiation dose) in one light irradiation differs for each individual. In addition, since the temperature of the LED light source gradually rises during one irradiation cycle and the apparent irradiance decreases, the longer the irradiation time is, the greater the irradiation dose error is. Therefore, an appropriate therapeutic effect cannot be achieved by using modifying method 1, in which the irradiation time is modified in view of the temperature of the LED light source at the start of irradiation.

As described above, by applying modifying method 2, in which the irradiation time is modified depending on the determined irradiation dose, the irradiation dose error can be made constant regardless of the determined irradiation dose.

As described above, in the embodiment, the irradiation time is modified depending on the temperature of the LED light source at the start of irradiation and is also modified depending on the determined irradiation dose, so that the same therapy apparatus can always provide stable therapeutic effects, even when the temperature conditions and irradiation dose setting conditions differ.

Variations

In the above-described embodiment, the change in temperature of the LED light source from the reference temperature is detected by measuring the temperature of the LED substrate in the measurement unit 21.

However, for monitoring the change in optical characteristics of the LED light source, it is also sufficient to detect another parameter that is correlated with the temperature of the LED light source. For example, the forward voltage Vf of the LED light source can be measured instead of the temperature of the LED substrate. There is a correlation between the temperature of the LED light source and the forward voltage Vf of the LED light source: when the temperature of the LED light source rises, the forward voltage Vf of the LED light source decreases. Therefore, measuring the forward voltage Vf of the LED light source can also be used for monitoring the change in optical characteristic.

Alternatively, instead of the temperature of the LED substrate, the characteristics of light emitted from the LED light source may be measured. The characteristics of light include spectral spectrum, irradiance, and radiant flux from the LED light source. In this case, the change in irradiance and the peak wavelength of the light emitted from the LED light source can be monitored directly.

In the above-described embodiment, the irradiation time of the light is modified in order to reduce the variation in the therapeutic effect caused by change in the optical characteristics of the LED light source.

However, the parameter for modifying the light irradiation dose is not limited to the irradiation time, and for example, the inputted current (forward current If) for the LED light source may be modified.

In addition, a temperature controller may be provided to control the temperature of the LED substrate in order to reduce variation in the therapeutic effect caused by change in the optical characteristics of the LED light source. For example, the temperature controller may include a fan with a variable rotation speed and/or a Peltier element. In this case, a temperature adjustment amount is calculated as the modifying value, and the temperature controller controls the temperature of the LED substrate on the basis of the calculated temperature adjustment amount.

In the above-described embodiment, the information shown in FIGS. 6B and 8B is stored in the storage unit 43 of the ultraviolet therapy apparatus 1, and the control unit 45 derives the modification factors α and β directly based on the temperature T of the LED substrate and the determined irradiation dose H. However, the first and second information is not limited to the information shown in FIGS. 6B and 8B, as long as information (first information) regarding the apparent irradiance used in modifying method 1 and information (second information) regarding the apparent irradiation dose used in modifying method 2 are stored in storage unit 43.

For example, information shown in FIGS. 6A and 8A may be stored in the storage unit 43. In this case, the control unit 45 derives the ratio of change in apparent irradiance from the information shown in FIG. 6A based on the temperature T of the LED substrate, and calculates the reciprocal of the ratio of change as the modification factor α. In addition, based on the determined irradiation dose H, the control unit 45 derives the ratio of the apparent irradiation dose and the determined irradiation dose from the information shown in FIG. 8A and calculates the reciprocal of the ratio as the modification factor β.

Other parameters that are useful for calculating the apparent irradiance (the spectral spectrum at the reference temperature and the erythema action spectrum) may be stored in the storage unit 43 as information (first information) regarding the apparent irradiance. In this case, using Formula (3) above, the control unit 45 calculates the apparent irradiance E′(T) at the temperature T of the LED substrate on the basis of the parameters stored in the storage unit 43 and the spectral spectrum at the temperature T of the LED substrate. The control unit 45 then calculates the irradiation time t by dividing the determined irradiation dose H by the apparent irradiance E′(T). This yields the same irradiation time (H/E×α) as that when the modification factor α is used.

Furthermore, the storage unit 43 may store information indicating the relationship between the irradiation time and the apparent irradiance, for example, as shown in FIG. 7, as information (second information) regarding the apparent irradiation dose. In this case, the control unit 45 calculates the apparent irradiance on the basis of the inputted determined irradiation dose with reference to the information stored in the storage unit 43, and calculates the modification factor β (the ratio of the determined irradiation dose to the apparent irradiation dose).

Furthermore, in the above-described embodiment, the temperature of the LED substrate is measured only at the start of irradiation and the irradiation dose is modified using both modifying method 1 and modifying method 2, but it is also possible to measure the temperature of the LED substrate during irradiation and to modify the irradiation dose in real time with use of modifying method 1. In this case, modifying method 2 may be unnecessary.

Modifying method 2 may also be omitted when the determined irradiation dose is small enough to allow for the irradiation dose error.

Furthermore, in the above-described embodiment, the light with the wavelength of 308 nm is used as the therapeutic light, but the wavelength of the therapeutic light can be set arbitrarily depending on the disease.

The ultraviolet therapy apparatus of the present invention is not limited to the above-described embodiment, and various changes can be made.

### REFERENCE SYMBOLS

1: Ultraviolet therapy apparatus, 2: Treatment tool, 21: Measurement unit, 4: Main unit, 41: Input unit, 42: Display unit, 43: Storage unit, 44: Power supply unit, 45: Control unit, 46: LED drive unit

1. An ultraviolet therapy apparatus, comprising:
an LED light source configured to emit light including ultraviolet light;
a controller configured to control lighting of the LED light source; and
a measurement unit configured to detect change in a temperature of the LED light source from a reference temperature,
the controller including:
a calculator configured to calculate a modifying value for modifying an irradiation dose of the ultraviolet light on the basis of change in a degree of effect on a human body caused by change in spectral spectrum of the light due to change in the temperature detected by the measurement unit, and
a lighting controller configured to cause the LED light source to emit the light on the basis of the modifying value calculated by the calculator.

2. The ultraviolet therapy apparatus according to claim 1, further comprising a storage unit for storing first information regarding an apparent irradiance that is obtained by modifying an irradiance on an irradiated surface of the ultraviolet light in view of an erythema action at each wavelength, wherein the calculator calculates the modifying value using the first information stored in the storage unit.

3. The ultraviolet therapy apparatus according to claim 2, wherein the first information is information that indicates a relationship between a parameter correlated with the temperature of the LED light source and the apparent irradiance, and wherein
the calculator is configured to
derive a first modification factor that is a value obtained by dividing the apparent irradiance at the reference temperature by the apparent irradiance corresponding to the temperature of the LED light source on the basis of the change in temperature detected by the measurement unit with reference to the first information stored in the storage unit, and
calculate the modifying value by multiplying a parameter that determines the irradiation dose of the ultraviolet light at the reference temperature by the first modification factor.

4. The ultraviolet therapy apparatus according to claim 2, wherein the first information is information that is useful for calculating the apparent irradiance and includes information on a spectral spectrum of the ultraviolet light and an erythema action spectrum, and wherein
the calculator is configured to
calculate the apparent irradiance at the temperature of the LED light source on the basis of the change in temperature detected by the measurement unit with reference to the first information stored in the storage unit, and
calculate the modifying value on the basis of the calculated apparent irradiance.

5. The ultraviolet therapy apparatus according to claim 1, comprising a storage unit for storing second information regarding an apparent irradiation dose derived from change in apparent irradiance during lighting of the LED light source, the apparent irradiance being obtained by modifying an irradiance on an irradiated surface of the ultraviolet light in view of an erythema action at each wavelength, wherein the calculator calculates the modifying value using the second information stored in the storage unit.

6. The ultraviolet therapy apparatus according to claim 5, wherein the second information is information that indicates a relationship between a determined irradiation dose to be applied to a patient and a ratio of the apparent irradiation dose to the determined irradiation dose, the ultraviolet therapy apparatus further comprising an input unit configured to input the determined irradiation dose, wherein
the calculator is configured to
derive a second modification factor that is a reciprocal of the ratio of the apparent irradiation dose to the determined irradiation dose on the basis of the determined irradiation dose inputted by the input unit with reference to the second information stored in the storage unit, and
calculate the modifying value by multiplying a parameter that determines the irradiation dose of the ultraviolet light by the second modification factor.

7. The ultraviolet therapy apparatus according to claim 1, wherein the measurement unit measures any one of a temperature of an LED substrate on which the LED light source is mounted, a forward voltage of the LED light source, and characteristics of the ultraviolet light from the LED light source.

8. The ultraviolet therapy apparatus according to claim 1, wherein the calculator calculates, as the modifying value, any one of an irradiation time of the ultraviolet light, the inputted current for the LED light source, and a temperature adjustment amount for the LED light source.

9. The ultraviolet therapy apparatus according to claim 1, wherein the measurement unit detects the change in temperature of the LED light source before the LED light source is turned on by the lighting controller,
wherein the calculator calculates an irradiation time of the ultraviolet light as the modifying value before the LED light source is turned on by the lighting controller, and
wherein the controller has a display controller configured to display the light irradiation time calculated by the calculator on a display unit before the LED light source is turned on by the lighting controller.

10. A method for applying ultraviolet light using an ultraviolet therapy apparatus having an LED light source that emits light including ultraviolet light, the method comprising:
a first step of detecting change in temperature of an LED light source from a reference temperature;
a second step of calculating a modifying value for modifying an irradiation dose of the ultraviolet light on the basis of change in a degree of effect on a human body caused by change in spectral spectrum of the light due to the change in temperature; and
a third step of causing the LED light source to emit on the basis of the modifying value.
